(19) United States
(12) Patent Application Publication
(10) Pub. No.: US 2013/0297039 A1
(43) Pub. Date: Nov. 7, 2013

(54) SAFETY STENT

(71) Applicant: UNIVERSITY OF ROCHESTER, Rochester, NY (US)

(72) Inventor: Granville L. LLOYD, Madison, WI (US)

(21) Appl. No.: 13/859,672

(22) Filed: Apr. 9, 2013

Related U.S. Application Data
(60) Provisional application No. 61/621,944, filed on Apr. 9, 2012.

(51) Int. Cl. A61F 2/04

(52) U.S. Cl. CPC A61F 2/042
USPC 623/23.66

(57) ABSTRACT

A stent can comprise a first elongate member and a second elongate member. The first elongate member can be configured to extend within a body lumen of a subject. The second elongate member can have (i) a first end portion connected to the first elongate member and (ii) a second end portion. An injection lumen can extend within the second elongate member for injecting a substance into at least a part of the body lumen.

### CROSS-REFERENCE TO RELATED APPLICATIONS

This application claims the priority benefit of U.S. Provisional Patent Application No. 61/621,944, filed Apr. 9, 2012, titled SAFETY STENT, the entire content of which is incorporated by reference herein.

### FIELD

This application relates generally to stents and associated methods and, in some embodiments, more particularly to ureteral stents and associated methods.

### BACKGROUND

Tubular prostheses, commonly referred to as stents, are used in a variety of medical procedures. For example, stents are sometimes used to assist drainage from the kidney through the ureter, from the liver through the biliary ducts, from the dorsal or ventral pancreas through the pancreatic ducts, from the gall bladder through the cystic, hepatic, or common bile ducts, and the like.

Stents are sometimes deployed in ducts is to provide drainage to circumvent a blockage. Blockage of ducts in the body can be a serious and very painful affliction that can result in death if not promptly and effectively treated. Blockages can occur for a number of reasons. For example, stones or debris from stones can pass from the kidney into the ureter, where they can become entrapped. Similarly, stones or debris can pass from the gall bladder into the bile ducts, where they become entrapped. Cysts and tumors growing against the outer wall of ducts can cause constriction of those ducts. Similarly, internal or duct wall cysts and tumors can act to block ducts.

One of the functions of ureteral stents includes bypassing ureteral obstruction and providing urinary drainage from the kidney to the bladder for a period of time, typically a few days to several months. The ureteral stent usually provides a drainage means such as a lumen for directing fluid from the renal pelvis to the bladder.

### SUMMARY

Ureteral stents can assist in detecting and addressing ureteral injury during complex retroperitoneal and pelvic surgery. The ureter is a delicate structure that can be involved in inflammatory masses or malignancies, or obscured by obesity. Many surgical cases, such as simple and radical hysterectomy, colon surgery for malignancy or inflammation, e.g., diverticulties, aneurysm, and many others, involve or pass very close to the ureter. Further, the ureter is often drawn into the inflammatory reaction present at such locations and can be encountered and injured in locales far from its anatomical “normal.”

Stents can be used to ensure patency of a ureter, which may be compromised by a kidney stone or other objects, so as to verify the integrity of the ureter during or at the conclusion of the above surgical procedures. This method is sometimes used as a temporary measure to prevent damage until additional procedures are performed.

However, currently, the pre-operative installation of a typical ureteral stent offers no easy way to test for ureteral integrity during and at the end of the surgical cases. This sometimes initiates the need to resort to emergency consultation of urologic specialist, which may be very time consuming, expensive, and often unavailable.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., Clause 1, Clause 10, Clause 19, Clause 24, Clause 25, Clause 34, or Clause 43. The other clauses can be presented in a similar manner.

1. A stent comprising:
a stent member comprising a first stent member end, a second stent member end, a first fluid port, a second fluid port, and a first lumen, the first fluid port being located at or near the first stent member end, the second fluid port being located at or near the second stent member end, the first lumen extending between and being in internal fluid communication with the first fluid port and the second fluid port, the stent member configured to extend within a body lumen between a first body portion and a second body portion of a subject;
an elongate injection member comprising a first injection member end portion and a second injection member end portion, the first injection member end portion being connected to the stent member, and the second injection member end portion comprising an injection port, an injection lumen extending within the elongate injection member for delivering a substance out of a third fluid port into at least a part of at least one of the body lumen, the first body portion, or second body portion.

2. The stent of Clause 1, wherein the injection lumen extends within the stent member, and the stent member comprises the third fluid port.

3. The stent of Clause 2, wherein the third fluid port is positioned at about ⅓ of the total length of the stent member from the first stent member end.

4. The stent of Clause 1, wherein the injection member is removably connected to the stent member.

5. The stent of Clause 1, wherein the second injection member end portion has a length sufficient to extend from the subject's urinary bladder to a location outside of the subject.

6. The stent of Clause 1, wherein the second injection member end portion is connected to a substance reservoir.

7. The stent of Clause 1, wherein the first lumen is dimensioned to allow a guidewire to extend therethrough.

8. The stent of Clause 1, wherein the substance includes a solution operative to show injury or integrity of the part of the body portion.

9. The stent of Clause 1, wherein the substance includes a contrast agent.

10. A stent comprising:
a first elongate member configured to extend within a body lumen of a subject; and
a second elongate member having (i) a first end portion connected to the first elongate member and (ii) a second end portion;
wherein an injection lumen extends within the second elongate member for injecting a substance into at least a part of the body lumen.

11. The stent of Clause 10, further comprising at least one outflow port through the first end portion of the second elongate member for injecting the substance into the part of the body lumen.

12. The stent of Clause 10, wherein the first elongate member defines a first lumen and a second lumen, the second lumen being separated from the first lumen within the elongate member such that fluid is not permitted to flow solely within the first elongate member between the first lumen in the second lumen, the first end portion of the injection lumen being in fluid communication with the second lumen.

13. The stent of Clause 12, further comprising an outflow port extending through a side wall of the first elongate member to inject the substance into the part of the body portion through the second lumen.

14. The stent of Clause 10, wherein the second elongate member has a length such that the second end portion of the second elongate member is accessible from outside of the subject while the first end portion is positioned within the subject's urinary bladder.

15. The stent of Clause 10, further comprising a separate injection tube configured to extend through a body wall of the subject to connect to the second end portion of the second elongate member.

16. The stent of Clause 10, wherein the first elongate member includes a first end portion configured to be retained at a kidney of the subject and a second end portion configured to be retained in the subject's urinary bladder while the first elongate member extends through the subject's ureter.

17. The stent of Clause 16, further configured such that the first end portion of the second elongate member and the first elongate member diverge in the bladder.

18. The stent of Clause 16, further configured such that the first end portion of the second elongate member and the first elongate member diverge in a renal pelvis of the subject.

19. A method of assessing a condition of a body lumen of a subject, the method comprising:
providing a stent comprising a first lumen, a second lumen, a first port, a second port, a third port, and a fourth port, the first lumen extending between the first port the second port, the second lumen extending between the second port in the third port, the third port being positioned between the first port and the second port;
positioning the stent at least partially within the subject such that the first lumen extends through the body lumen, the first port is positioned in a first body cavity, the second port is positioned in a second body cavity, the third port is positioned in the body lumen, and the fourth port is positioned outside of the first body cavity, the second body cavity, and the lumen;
connecting a reservoir containing a contrast agent to the fourth port;
injecting the contrast agent into the body lumen through fourth port, the second lumen, and the third port; and
obtaining images of the body lumen.

20. The method of Clause 21, wherein positioning the stent comprises positioning the fourth port outside of the subject.

21. The method of Clause 21, further comprising connecting the fourth port to a separate injection tube extending through a body wall of the subject.

22. The method of Clause 21, further comprising separating a portion of the stent from a remainder of the stent injection of the contrast agent, and removing a portion from the subject while leaving the remainder within the body, the portion comprising the fourth port and a first segment of the second lumen, the remainder comprising the first port, the second port, the entire first lumen, and second segment of the second lumen.

23. The method of Clause 22, when separating the portion from the remainder of the stent comprises cutting the stent.

24. A stent comprising:
a stent member comprising a first stent member end, a second stent member end, a first fluid port, a second fluid port, a third fluid port, a first lumen, and a second lumen, the first fluid port being located at or near the first stent member end, the second fluid port being located at or near the second stent member end, the first lumen extending between and being in internal fluid communication with the first fluid port and the second fluid port, the second lumen being in internal fluid communication with the third fluid port, the stent member configured to extend within a body lumen between a first body portion and a second body portion of a subject;
an elongate injection member comprising an injection lumen and having a first injection member end portion connected to the stent member such that the injection lumen is in internal fluid communication with the second lumen for delivering a substance through the injection lumen into the second lumen.

25. A stent deployable into a subject, comprising:
a stent member configured to extend within a body lumen that extends between a first body portion and a second body portion of the subject, the stent member defining a first lumen;
an elongate injection member having a proximal end portion connected to the stent member, the injection member defining an injection lumen in fluid communication with a second lumen for delivering a substance into the second lumen, the second lumen parallel to and fluidly isolated from the first lumen; and
at least one outflow port extending through a wall defining the second lumen for injecting the substance into at least a part of the body lumen, the first body portion, or second body portion.

26. The stent of Clause 25, wherein the stent member defines the second lumen.

27. The stent of Clause 26, wherein the outflow port is positioned at about ⅓ of the length of the stent member from an end of the stent member proximal to the injection member thereof.

28. The stent of Clause 25, wherein the injection member is removably connected to the stent member.

29. The stent of Clause 25, wherein the injection member has a distal end accessible outside of the subject.

30. The stent of Clause 25, wherein the injection member has a distal end connected to a source of the substance.

31. The stent of Clause 25, wherein the first lumen is sufficiently wide to allow a guidewire to extend therethrough.

32. The stent of Clause 25, wherein the substance includes a solution operative to show injury or integrity of the part of the body portion.

33. The stent of Clause 25, wherein the substance includes visual or radiographic solution.

34. A stent deployable into a body of a subject, comprising:
a first elongate member configured to extend within a body lumen of the subject; and
a second elongate member having (i) a proximal end portion connected to the first elongate member and (ii) a distal end portion;
wherein the second elongate member defines an injection lumen for injecting a substance into at least a part of the body lumen through the proximal end portion thereof.

35. The stent of Clause 34, further comprising at least one outflow port perforated through the proximal end portion of the second elongate member for injecting the substance into the part of the body lumen.

36. The stent of Clause 34, wherein the first elongate member defines a first lumen and a second lumen isolated from the first lumen, the proximal end portion of the injection lumen being merged with the second lumen.

37. The stent of Clause 34, further comprising an outflow port extending through a side wall of the first elongate member to inject the substance into the part of the body portion through the second lumen.

38. The stent of Clause 34, wherein the distal end portion of the second elongate member is accessible from outside of the subject.

39. The stent of Clause 34, further comprising a separate injection tube extending through a body wall of the subject to connect the distal end portion of the second elongate member.

40. The stent of Clause 34, wherein the first elongate member includes a proximal end portion configured to be retained at a kidney of the subject and a distal end portion configured to be retained in a bladder of the subject while being deployed to extend through a ureter of the subject.

41. The stent of Clause 40, wherein the proximal end portion of the second elongate member connects to the first elongate member in the bladder.

42. The stent of Clause 40, wherein the proximal end portion of the second elongate member connects to the first elongate member in a renal pelvis of the subject.

43. A method of monitoring condition of a body lumen of a subject, comprising:
installing an elongate tubular member within the body lumen, the elongate tubular member providing a first fluid passageway extending through the body lumen and a second fluid passageway extending through a part of the body lumen;
connecting the second fluid passageway with an injection arm, wherein the injection arm defines a fluid passageway in fluid connection with the second fluid passageways of the elongate tubular member; and
injecting, from the fluid passageway of the injection arm, a visual or radiographic solution into the body lumen through the second fluid passageway.

44. The method of Clause 43, further comprising supplying the solution from a distal end of the injection arm from outside of the body lumen.

45. The method of Clause 43, further comprising connecting the injection arm with a separate injection tube extending through a body wall of the subject.

46. The method of Clause 43, further comprising removing the injection arm from the elongate tubular member after the visual or radiographic solution has been injected or the elongate tubular member is installed.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 is a schematic drawing showing an external configuration of an exemplary stent according to an embodiment.

FIG. 2 illustrates an exemplary stent applied to a ureter of a patient according to an embodiment.

FIG. 3 illustrates regions of an elongate stent member of an exemplary stent according to an embodiment.

FIG. 4 illustrates, according to an embodiment, internal structure of an elongate stent member and an injection arm connected to the elongate stent member.

FIG. 5 is a cross-sectional view of the elongate stent member taken along line A shown in FIG. 3.

FIG. 6 is a cross-sectional view of the elongate stent member taken along line B shown in FIG. 3.

FIG. 7 illustrates an exemplary stent according to an embodiment.

FIG. 8 illustrates an application, according to an embodiment, of the safety stent of FIG. 7.

### DETAILED DESCRIPTION

In the following detailed description, numerous specific details are set forth to provide a full understanding of the subject technology. It will be apparent, however, to one of ordinarily skilled in the art that the subject technology may be practiced without some of these specific details. In other instances, well-known structures and techniques have not been shown in detail so as not to obscure the subject technology.

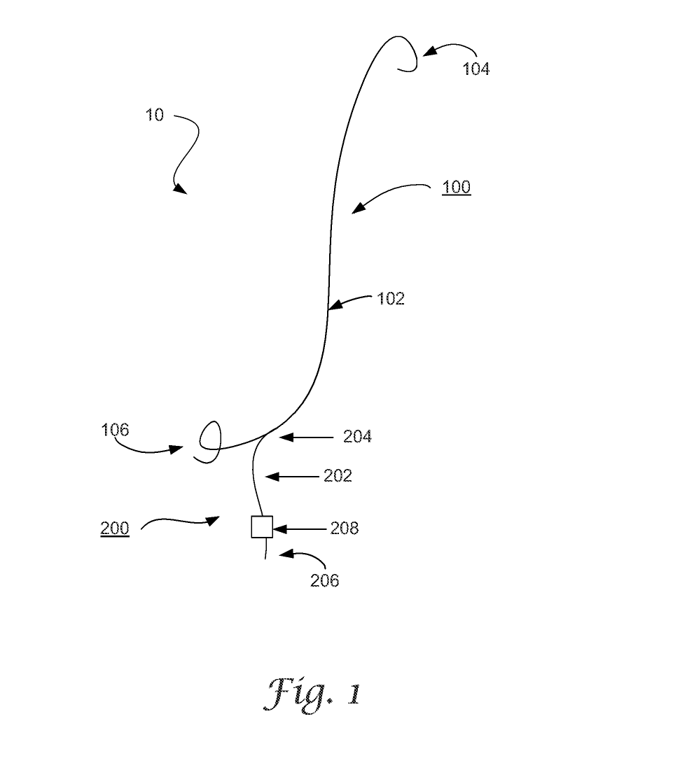

FIG. 1 is a schematic drawing of a stent 10 in accordance with an embodiment. The stent 10 comprises an elongate stent member 100. The elongate stent elongate stent member 100 comprises a first end portion 104 and a second end portion 106. The elongate stent member 100 can further include an elongate tubular body portion 102 extending between the first and the portion 104 and the second end portion 106. In some embodiments, a portion of the elongate tubular body portion 102 can be substantially straight in the absence of external forces.

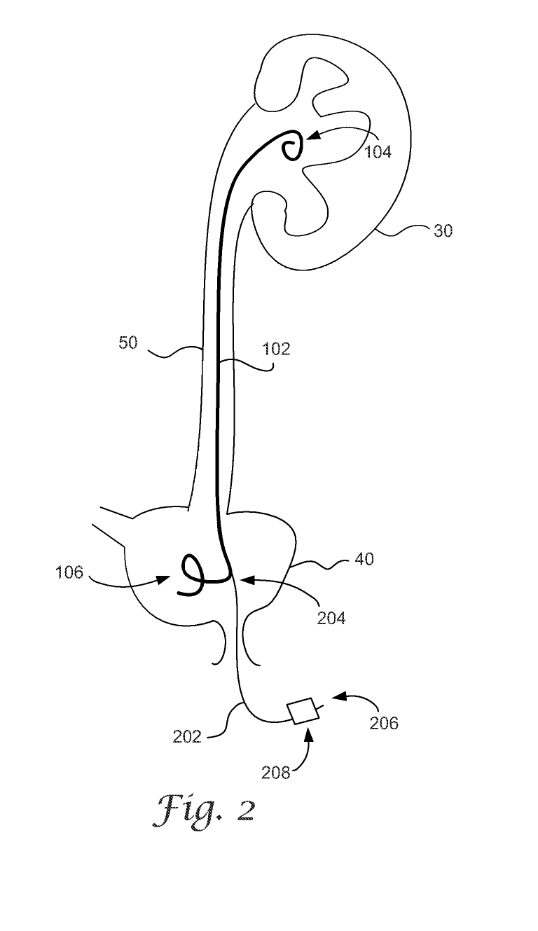

The first and second end portions 104 and 106 can be configured to be retained in or secured within intended organs or body tissues of a patient to whom the stent 10 is applied. For example, the first and second end portions 104 and 106 can be configured with hooks, loops or any anchoring or retaining structure such that after deployment, the stent 10 will be retained within the desired location inside of the patient's body. In some embodiments, when the stent is used as a ureteral stent, the first end portion 104 of the stent 10 can be curved and secured within the kidney 30 and the second end portion 106 can be curved and secured within the bladder 40 after deployment. In this way (for example), the tubular body portion 102 can extend along the ureter 50 between the kidney 30 and the urinary bladder 40 as shown in FIG. 2 for example.

The stent 10 can further comprise an injection arm 200. For example, as shown in FIGS. 1 and 2, the injection arm 200, also referred to herein as an elongate injection member and an injection stent member, can include an elongate tubular portion 202 branching from a part of the elongate tubular body portion 102. In some embodiments, the tubular portion 202 branches from the tubular portion 102 in proximity to the second end portion 106. The elongate tubular portion 202 can include a first end 204 and a second end 206. The first end 204 can be joined with the tubular member 102.

The length of the elongate tubular portion 202 can vary in accordance with the patient's condition and characteristics, e.g., the gender and/or age. For example, a longer elongate tubular portion 202 may be required for a stent 10 installed in a male patient than a stent installed in a female patient.

In some embodiments, the second end 206 can include an adapter or a connector 208 that can be connected a solution reservoir. For example, the adapter 208 can include a luer-lock adapter or an adapter that fits with a syringe. The function of the adapter 208 is further discussed herein below.

In addition or alternative to the adapter 208, the second end 206 of the injection arm 200 can include a free end allowing a user to assess and control the deployment and removal of the injection arm 200. For example, the elongate stent member 100 and the safety injection arm 200 can be so configured that the stent 10 can be removed from the patient simply by pulling the second end 206 of the safety injection arm 200. In some embodiments, the safety injection arm 200 can be detached from the elongate stent member 100 after the stent 10 has been deployed. As illustrated schematically in FIG. 2, the second end 206 can extend outside of the patient's body.

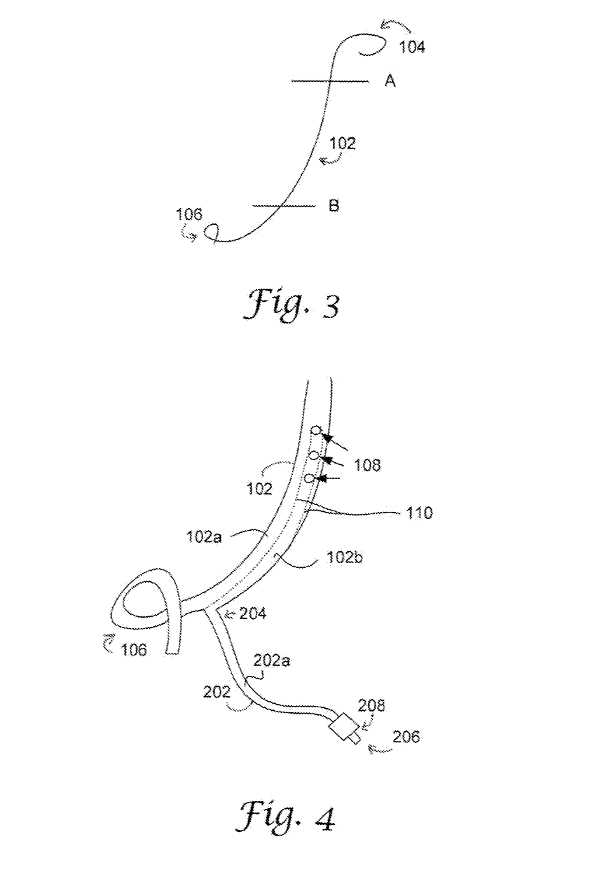

As shown in FIG. 3, the elongate tubular member 102 of the stent 10 can be virtually divided into three regions. For example, the three regions can include a major portion of the elongate tubular body portion 102 between reference lines A and B, a first end portion above line A (near the first end 104), and a second end portion below line B (near the second end 106). Exemplary internal structures of the elongate tubular body portion 102 extending through these regions are further illustrated in FIG. 4.

As illustrated in FIG. 4, the elongate tubular body portion 102 can comprise two lumens 102a, 102b in the region between lines A and B. In some embodiments, portions of the lumens 102a, 102b can extend parallel to each other in the tubular body portion 102. The lumen 102a can extend from the second end 106 to the first end 104. The lumen 102b can be in fluid communication with the elongate tubular portion 202 of the injection arm 200. The elongate tubular body portion 102 can further include at least one outflow port or orifice 108.

In the embodiment illustrated in FIG. 4, three outflow ports or orifices 108 are formed through the sidewall of the elongate tubular body portion 102. Although FIG. 4 illustrates three outflow ports or orifices 108, other numbers of ports or orifices can be used in some embodiments. For example, a stent 10 can comprise a single outflow port or orifice 108 in some embodiments.

The outflow ports or orifices 108 can be positioned along the tubular body portion 102. The outflow ports or orifices 108 can be positioned at or near an end of the tubular body portion 102 or between the ends of the tubular body portion 102 various embodiments. When the stent 10 is applied as a ureter stent, the orifices 108 can be positioned, for example, at a location ⅓ of the total length of the elongate tubular body portion 102 from the second end 106. In some embodiments, the orifices 108 can be spaced from the first end 104 or the second end 106 by a distance that is ¼ to ½ of the total length of the elongate tubular body portion 102. In some embodiments, the orifices 108 can be spaced from the first end 104 or the second end 106 by a distance that is ¼, ⅓, or ½ of the total length of the elongate tubular body portion 102. In some embodiments, the distance between the second end 106 and the outflow ports 108 can be about 8 cm. In some embodiments, the distance between the second end 106 and the outflow ports 108 can be 6 to 10 cm, 4 to 12 cm, or 2 to 14 cm. This distance can reflect the commonly injured region of ureter during installation of the stent 10 or at any stage of a surgical case.

With continued reference to FIG. 4, in some embodiments, the elongate tubular portion 202 can also define a lumen 202a extending between the first end 204 and the second end 206. The lumen 102b can be in fluid communication with the lumen 202a and thus establish a passageway for the fluid, e.g., injected from the adapter 208, to flow through the elongate tubular body portion 102 and into the body lumen of the patient from the outflow ports 108. The lumen 102b and the lumen 202a can be considered to form a single lumen extending within both the elongate tubular portion 202 and the tubular body portion 102.

Various fluids and/or solutions can be introduced into the injection arm 200 through the adapter 208. In one embodiment, the second end 206 of the safety arm 200 can have a transverse dimension of about 5 French and can be configured to be pushed into a tapered female tip that screws into the syringe or luer-lock adapter. In some embodiments the fluids and/or solutions can include, for example, contrast agents, native tissue fluorescence substrates, or visual chromogens such as Methlyene Blue. Suitable contrast agents can include radiographic contrast agents. The fluids and/or solutions can be selected to demonstrate integrity or injury using X-ray or other imaging modalities. When the stent 10 is used as a ureteral stent to extend through a patient's ureter, injection of imaging agents can be used to assess the condition of the ureter using medical imaging techniques.

As discussed above, a fluid/solution can be directed into the elongate tubular portion 202 and eventually injected into the ureter of the patient through the outflow ports 108. In such embodiments, the patency of the ureter, as well as the integrity or any injury or damage thereof, can be monitored or evaluated during or at the conclusion of any surgical cases without the need of the presence of a urological specialist. In some embodiments, the adapter 208 can be any kind of adapter or valve for controlling the quantity and flow rate of solution injecting or flowing into the stent 10.

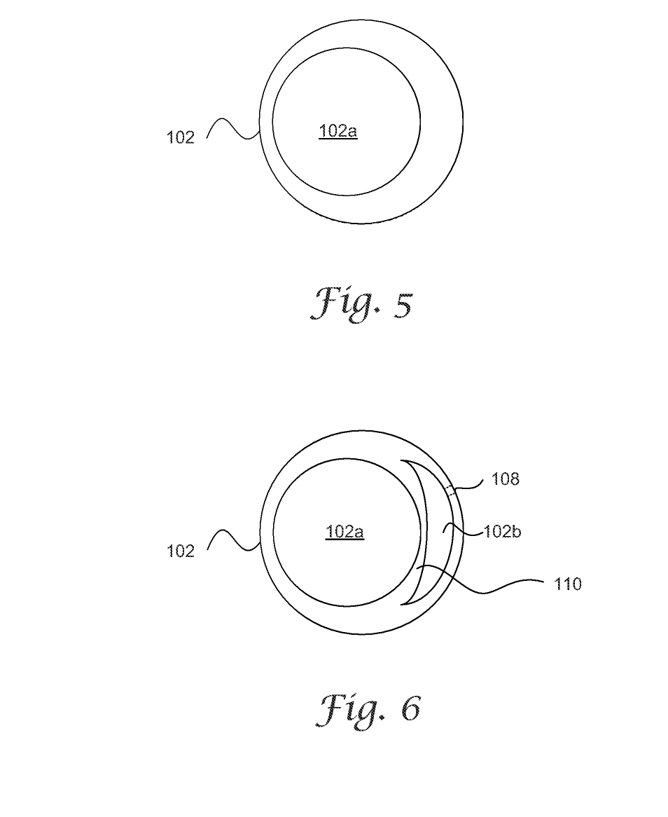

FIG. 5 is a cross-sectional view of the elongate tubular portion 102 taken along line A shown in FIG. 3. FIG. 6 is a cross-sectional view of the elongate tubular portion 102 taken along line B shown in FIG. 3.

As shown in FIG. 5, the elongate tubular portion 102 can have a single lumen 102a at the location of line A. In some embodiments for use in the urinary system, the lumen 102a can have a diameter sufficient for accommodating a guidewire or delivery wire to aid proper installation of the stent 10 with the first and second end portions 104 and 106 anchored at the kidney and the urinary bladder, respectively. The lumen 102a can function as would the single lumen of a conventional ureteral stent, e.g., bypassing ureteral obstruction and providing urinary drainage from the kidney to the bladder. In some embodiments, the elongate tubular portion 102 can have a diameter at the location of line A that is no larger than 4.5 French (FR), with the lumen 102a configured to allow a guide wire to extend through the lumen 102a.

As shown in FIG. 6, the elongate tubular portion 102 can have two lumens 102a, 102b at the location of line B. The lumens 102a and 102b can be separated by a wall 110 within the tubular portion 102. The functions and dimensions of the lumen 102a at line B is similar to that at line A. With the inclusion of the lumen 102b, the cross-sectional dimensions of the elongate tubular portion 102 at line B can be, in some embodiments, no larger than about 6.7 FR, for example. As shown in FIG. 6, the wall 110 separates the lumen 102a from the lumen 102b such that the lumen 102a and the lumen 102b are not in fluid communication via a path that is solely within the tubular portion 102. It will be appreciated that, under certain conditions or requirements, the lumen 102b can be designed to be in fluid communication with the lumen 102a via a path that is solely within the tubular portion 102. This alternate design does not extend beyond the scope of the current application.

In some embodiments, the stent 10 can be fabricated from polyurethane, silicone, thermoplastic elastomers, or other materials known to those of skill the art. Materials may be selected by those of skill in the art to provide suitable strength, flexibility, low friction, biodurability, biocompatibility, and reasonable cost.

Referring again to FIG. 2, in some embodiments, the stent 10 can be placed as shown in FIG. 2 by positioning a guidewire through a ureter of interest, then advancing stent 10 over the guide wire followed by removal of the guide wire. The stent can be positioned such that the tubular body portion 102 extends through the ureter, the first end portion 104 is positioned at the kidney, the second end portion is positioned in the urinary bladder, the outflow port(s) 108 is/are positioned in the ureter, and the adapter 208 is positioned outside of the kidney, urinary bladder, and the ureter. The adapter 208 can be connected to a reservoir for a fluid and/or substance. The fluid and/or substance can be injected into at least one of the ureter, kidney, or urinary bladder through the outflow port(s) 108. Images of the area of interest may be obtained to aid assessment.

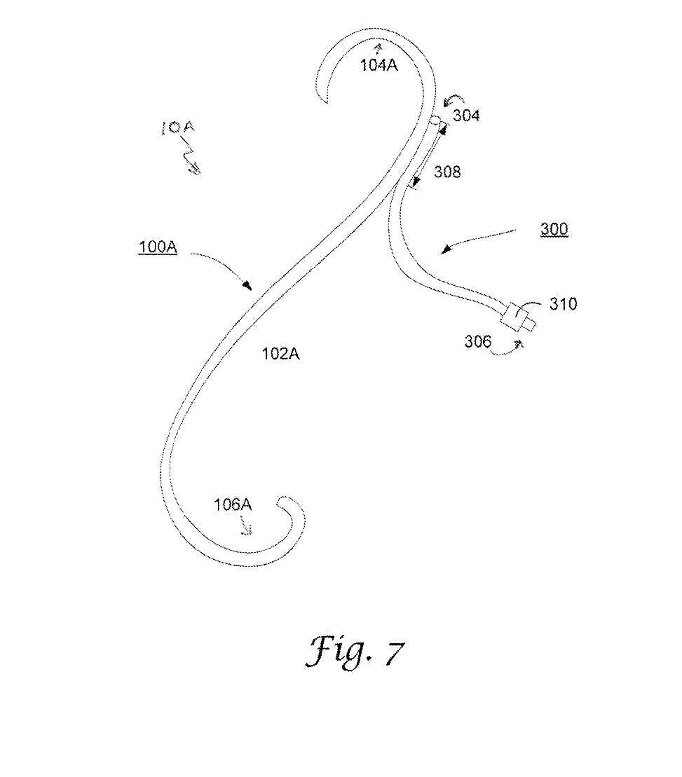

FIG. 7 shows an exemplary stent 10A that can be used for pyeloplasty repair and other operations in both adults and children. The stent 10A can be pre-operatively deployed in the ureter in a way similar to the stent 10, shown in FIGS. 1-6. Some components of the stent 10A are similar to corresponding components of the stent 10 described with reference to FIGS. 1-6 and are indicated by similar references numerals with the suffix “A.” For brevity, a detailed description of those components is not repeated.

According to some embodiments, the stent 10A includes an elongate stent member 100A and a side arm 300. The elongate stent member 100A includes a tubular elongate member 102A extending between a first end portion 104A and a second end portion 106A. The side arm 300 includes a first end 304 and a second end 306. As illustrated in FIG. 7, the first end 304 can be attached to the first end portion 104A of the elongate stent member 100A. An efflux or injection site 308 is formed along the region of attachment between the elongate stent member 100A and the side arm 300. The efflux or injection site 308 includes at least one outflow port similar to the outflow ports 108 shown in FIG. 4. The length of the attachment can be about 0.5 cm to about 3 cm, for example. It is appreciated that the length of the attachment can be varied according to specific conditions of the patient. The second end 306 of the side arm 300 can be connected to an adapter 310. The adapter 310 can be similar to the adapter 208.

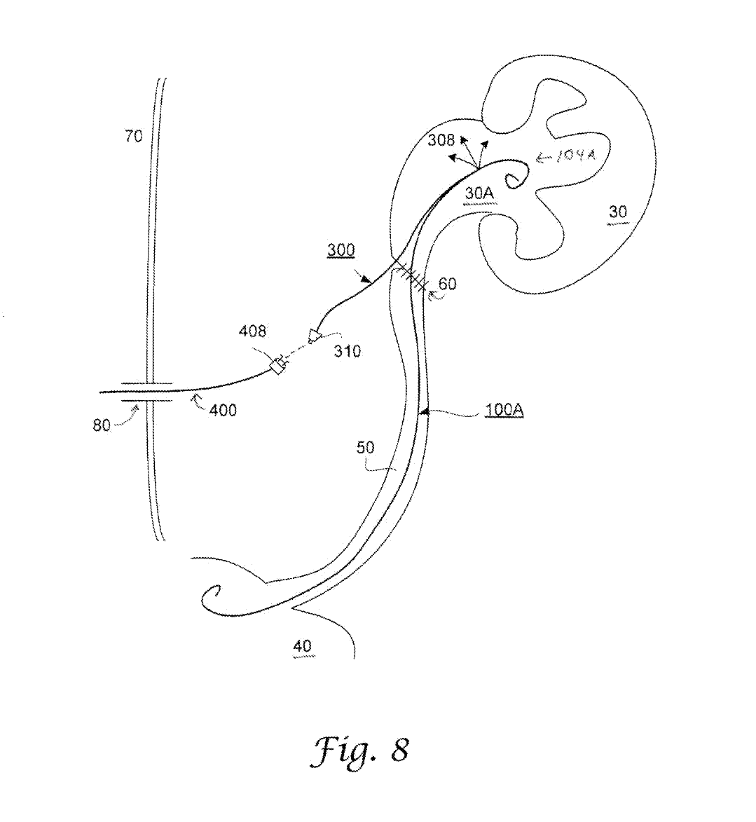

FIG. 8 shows an application of the stent 10A according to some embodiments. The stent 10A can be deployed into the ureter 50 in a similar to deployment of the stent 10 shown in FIGS. 1-6.

As shown in FIG. 8, the first end portion 104A of the elongate tubular member 100A can extend to the kidney 30. As such the efflux sites 308 can be located at or near the kidney 30. For example, the efflux sites 308 can be located at the renal pelvis 30A.

The side arm 300 can be accessed by connecting a separate injection tube 400 via the adapter 310 and an adapter 408. The injection tube 400 can extend through an open incision or a port 80 of the body wall 70 during laparoscopic or robotic surgery.

By injecting fluids and/or solutions (such as those described above) through the side arm 300, an individual (e.g., a surgeon) can effectively complete the anastomosis except for the last stitch 60, hold the suture around the efflux site 308, and test the integrity of the repair using the injection.

Once the anastomosis is verified as being watertight, the side arm 300 can be removed or cut away followed by placement and/or tightening of the final stitch 60, while the elongate stent member 100A can be left within the ureter 50. In some embodiments, the stent 10A can allow the surgeon to omit the drain placement which is associated with additional pain after pyeloplasty.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. A stent comprising:
a stent member comprising a first stent member end, a second stent member end, a first fluid port, a second fluid port, and a first lumen, the first fluid port being located at or near the first stent member end, the second fluid port being located at or near the second stent member end, the first lumen extending between and being in internal fluid communication with the first fluid port and the second fluid port, the stent member configured to extend within a body lumen between a first body portion and a second body portion of a subject;
an elongate injection member comprising a first injection member end portion and a second injection member end portion, the first injection member end portion being connected to the stent member, and the second injection member end portion comprising an injection port, an injection lumen extending within the elongate injection member for delivering a substance out of a third fluid port into at least a part of at least one of the body lumen, the first body portion, or second body portion.

2. The stent of claim 1, wherein the injection lumen extends within the stent member, and the stent member comprises the third fluid port.

3. The stent of claim 2, wherein the third fluid port is positioned at about ⅓ of the total length of the stent member from the first stent member end.

4. The stent of claim 1, wherein the injection member is removably connected to the stent member.

5. The stent of claim 1, wherein the second injection member end portion has a length sufficient to extend from the subject's urinary bladder to a location outside of the subject.

6. The stent of claim 1, wherein the second injection member end portion is connected to a substance reservoir.

7. The stent of claim 1, wherein the first lumen is dimensioned to allow a guidewire to extend therethrough.

8. The stent of claim 1, wherein the substance includes a solution operative to show injury or integrity of the part of the body portion.

9. The stent of claim 1, wherein the substance includes a contrast agent.

10. A stent comprising:
a first elongate member configured to extend within a body lumen of a subject; and
a second elongate member having (i) a first end portion connected to the first elongate member and (ii) a second end portion;
wherein an injection lumen extends within the second elongate member for injecting a substance into at least a part of the body lumen.

11. The stent of claim 10, further comprising at least one outflow port through the first end portion of the second elongate member for injecting the substance into the part of the body lumen.

12. The stent of claim 10, wherein the first elongate member defines a first lumen and a second lumen, the second lumen being separated from the first lumen within the elongate member such that fluid is not permitted to flow solely within the first elongate member between the first lumen in the second lumen, the first end portion of the injection lumen being in fluid communication with the second lumen.

13. The stent of claim 12, further comprising an outflow port extending through a side wall of the first elongate member to inject the substance into the part of the body portion through the second lumen.

14. The stent of claim 10, wherein the second elongate member has a length such that the second end portion of the second elongate member is accessible from outside of the subject while the first end portion is positioned within the subject's urinary bladder.

15. The stent of claim 10, further comprising a separate injection tube configured to extend through a body wall of the subject to connect to the second end portion of the second elongate member.

16. The stent of claim 10, wherein the first elongate member includes a first end portion configured to be retained at a kidney of the subject and a second end portion configured to be retained in the subject's urinary bladder while the first elongate member extends through the subject's ureter.

17. The stent of claim 16, further configured such that the first end portion of the second elongate member and the first elongate member diverge in the bladder.

18. The stent of claim 16, further configured such that the first end portion of the second elongate member and the first elongate member diverge in a renal pelvis of the subject.

19. A method of assessing a condition of a body lumen of a subject, the method comprising:
providing a stent comprising a first lumen, a second lumen, a first port, a second port, a third port, and a fourth port, the first lumen extending between the first port the second port, the second lumen extending between the second port in the third port, the third port being positioned between the first port and the second port;
positioning the stent at least partially within the subject such that the first lumen extends through the body lumen, the first port is positioned in a first body cavity, the second port is positioned in a second body cavity, the third port is positioned in the body lumen, and the fourth port is positioned outside of the first body cavity, the second body cavity, and the lumen;
connecting a reservoir containing a contrast agent to the fourth port;
injecting the contrast agent into the body lumen through fourth port, the second lumen, and the third port; and
obtaining images of the body lumen.

20. The method of claim 19, wherein positioning the stent comprises positioning the fourth port outside of the subject.
